(19) United States
(12) Patent Application Publication
(10) Pub. No.: US 2023/0240973 A1
(43) Pub. Date: Aug. 3, 2023

(54) CANNABIS SATIVA EXTRACTS AND THEIR USES

(71) Applicant: LINNEA SA, Riazzino (Locarno) (CH)

(72) Inventors: Nora ROSSINI, Riazzino (Locarno) (CH); Mario DELL'AGLI, Milano (IT); Marcello LUZZANI, Riazzino (Locarno) (CH); Giuseppe PALADINO, Riazzino (Locarno) (CH); Umberto CIRIELLO, Riazzino (Locarno) (CH); Enrico SANGIOVANNI, Milano (IT); Stefano PIAZZA, Milano (IT); Selina RUSCONI, Riazzino (Locarno) (CH)

(73) Assignee: LINNEA SA, Riazzino (Locarno) (CH)

(21) Appl. No.: 18/004,945

(22) PCT Filed: Jul. 13, 2021
(86) PCT No.: PCT/IB2021/056291

§ 371 Date: Jan. 10, 2023

(30) Foreign Application Priority Data
Jul. 15, 2020 (IT) 102020000017137

(51) Int. Cl. A61K 8/9789; A61Q 19/00; A61K 31/00; A61K 36/185; A61P 29/00

(52) U.S. Cl. CPC A61K 8/9789; A61Q 19/00; A61K 31/658; A61K 36/185; A61P 29/00; A61K 2800/57

(57) ABSTRACT

A composition of a Cannabis sativa extract comprising cannabinoids in concentrations ranging from 0.500 to 10.000% (w/w), and terpenes in concentrations ranging from 0.005 to 1.000% (w/w). The use of the composition in the prevention and/or treatment of inflammatory diseases is also described. The composition may also be used as a functional food ingredient.

The present invention relates to a composition which is a Cannabis sativa extract and its use in the prevention and/or treatment of inflammatory pathologies. A further object of the present invention is the use of said composition as a functional food ingredient.

### STATE OF THE ART

Inflammatory processes in the human body are treated with steroids or non-steroidal active ingredients. A further treatment involves the use of natural extracts.

Hemp oil, obtained mainly from hemp seeds (Cannabis sativa) is known for its anti-inflammatory properties. Contains a balanced combination of saturated and unsaturated fatty acids. Unsaturated fatty acids are precursors of arachidonic acid and indirectly influence inflammatory processes. Metabolites of unsaturated fatty acids positively affect skin functions, such as cell nutrition and skin revitalization, thus preventing the development of skin diseases. However, the anti-inflammatory effect of unsaturated fatty acids and their metabolites is low. Hemp oil also contains cannabidiol (CBD), a compound known for its anti-inflammatory properties. However, the concentration of CBD in currently available hemp oils is too low to exert an anti-inflammatory effect (Marcel O. Bonn-Miller et al., Journal of the American Medical Association, 2017, 318 (17), 1708-1709).

A further problem that is typically observed using natural extracts is the poor reproducibility of the observed activity, linked to the high variability that is typically found in the qualitative-quantitative composition between extracts from different batches (Dan Jin et al., Scientific Reports (2020) 10: 3309).

There is a strongly felt need to have a characterized and standardized extract of Cannabis sativa with high anti-inflammatory activity.

### DESCRIPTION OF THE INVENTION

Through a field selection process, the authors of the present invention have identified some varieties of Cannabis sativa with an average THC content of less than 0.2%, which, grown under defined conditions, are able to originate an extract that has a defined and reproducible component in cannabinoids and terpenes.

This extract has excellent anti-inflammatory activity, with excellent reproducibility between different batches.

### FIGURE DESCRIPTION

FIG. 1: toxicity of the composition according to the present invention evaluated on HaCaT cells after (A) 6 hours or (B) 24 hours of treatment with the extract according to the present invention in MCT (C. sativa extract in MCT) at the indicated doses, expressed as cell viability % (% viability).

FIG. 2: toxicity of the composition according to the present invention evaluated on HDF cells after (A) 6 hours or (B) 24 hours of treatment.

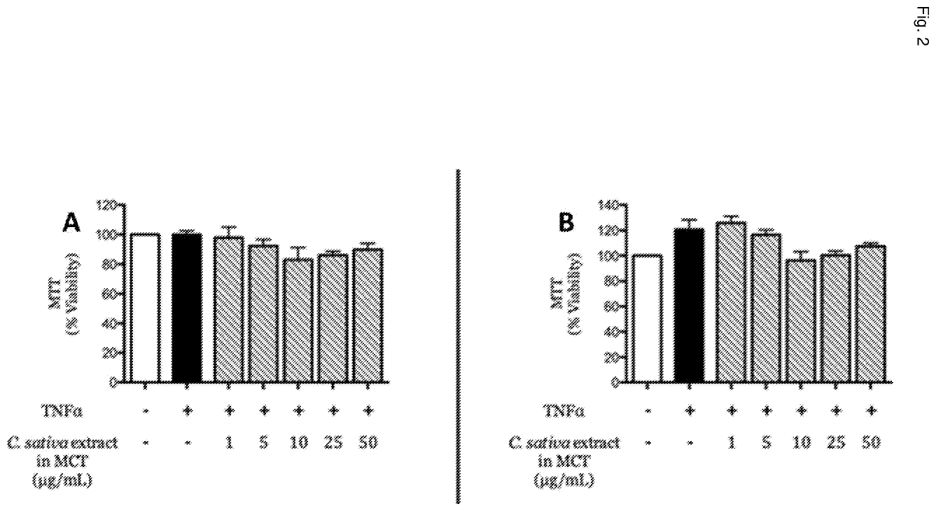

FIG. 3: comparative; pure CBD (Cannabidiol) toxicity evaluated on HDF cells after (A) 6 hours or (B) 24 hours of treatment.

FIG. 4: comparative; pure CBD toxicity assessed on HaCaT cells after (A) 6 hours or (B) 24 hours of treatment.

FIG. 5: effect (A) on NF-kB driven transcription and secretion of (B) IL8, (C) VEGF, (D) MMP-9 evaluated on HaCaT cells stimulated with TNF-α upon exposure to the indicated doses of the composition according to the present invention. The values are expressed as % versus the values measured in the presence of TNFα alone.

FIG. 6: comparative; effect (A) on NF-kB-induced transcription and release of (B) IL8, (C) VEGF, (D) MMP-9 evaluated on TNF-α-stimulated HaCaT cells following exposure to the indicated doses of CBD pure.

FIG. 7: effect evaluated on TNF-α stimulated HDF cells (A) on transcription induced by NF-kB and on release of (B) IL8, (C) MMP-9 following exposure to the indicated doses of the composition according to the present invention and, for comparative purposes, (D) on NF-kB-induced transcription and (E) IL8, (F) MMP-9 release following exposure to pure CBD.

### DETAILED DESCRIPTION OF THE INVENTION

The present invention relates to a composition comprising cannabinoids in concentrations ranging from 0.500 to 10.000% or between 0.500 and 7.000% (w/w), and terpenes in concentrations ranging from 0.005 to 1.000% or between 0.005 and 0.500% (w/w).

Said composition is a standardized extract prepared starting from aerial parts of Cannabis sativa L. The cannabinoids, in fact, accumulate in particular structures present on the surface of the Cannabis sativa plant, the glandular trichomes. The presence of these trichomes is proportional to the presence of cannabinoids and are more concentrated in the upper parts, particularly in bracts, flowers and small leaves.

In preferred embodiments, the varieties of Cannabis sativa L. used have an average THC content of less than 0.2%, measured in the dried mature inflorescences. Preferably, said varieties are Kompolti and Carmagnola.

The selected parts of the plant are treated by extraction with solvent, preferably ethanol, followed by heating in order to decarboxylate the acid forms of the cannabinoids into neutral cannabinoids. It is in fact known in the art that the acid form of cannabinoids is less active from a biological point of view and less stable from a chemical point of view: thermal decarboxylation allows to standardize and stabilize the chemical composition of the extract.

The resulting extract is therefore a mixture of cannabinoids, mainly cannabidiol, natural terpenes, and mainly lipophilic substances naturally occurring in Cannabis sativa L.

In preferred embodiments, the extract described above is further treated by removing the solvent and replacing it with an inert oily vehicle. Preferably, this inert oily vehicle is represented by a mixture of medium chain triglycerides (MCT).

Preferably, said composition comprises cannabinoids in concentrations ranging from 4.000 to 7.000% or from 4.100 to 6.500% (w/w), and terpenes in concentrations ranging from 0.050 to 0.300% or between 0.100 and 0.250% (w/w).

Advantageously, said composition comprises from 0.5 to 6.9% (w/w) of cannabidiol (CBD), from 0.1 to 3% (w/w) of other cannabinoids other than CBD, and from 0.005 to 0.5% (w/w) of terpenes.

Preferably, said composition comprises from 4 to 6% (w/w) of cannabidiol (CBD), from 0.2 to 2% (w/w) of other cannabinoids other than CBD, and from 0.05 to 0.3% (p/p) of terpenes.

In a preferred form, said cannabinoids comprise: cannabidiol (CBD), cannabigerol (CBG), cannabichromene (CBC), tetrahydrocannabinol (THC). In an even more preferred form, they also comprise: cannabidivarin (CBDV), cannabidiolic acid (CBDA), cannabinol (CBN), cannabidiol-C4 (CBD-C4).

In a preferred form, said terpenes comprise: beta-caryophyllene, alpha-humulene, farnesene isomer 3, farnesene isomer 2, farnesene isomer 1, caryophyllene oxide, and alpha-bisabolol.

In a preferred form, said composition comprises:

cannabidiol (CBD) 0.500-6.950% (w/w);

cannabigerol (CBG) 0.010-0.250% (w/w);

cannabichromene (CBC) 0.018-0.300% (w/w);

tetrahydrocannabinol (THC) 0.010-0.240% (w/w);

cannabidivarin (CBDV) 0.001-0.070% (w/w);

cannabidiolic acid (CBDA) 0.001-0.100% (w/w);

cannabinol (CBN) 0.001-0.070% (w/w);

cannabidiol-C4 (CBD-C4) 0.001-0.070% (w/w);

beta-caryophyllene 0.001-0.150% (w/w);

alpha-humulene 0.001-0.150% (w/w);

farnesene isomer 3 0.001-0.150% (w/w);

farnesene isomer 2 0.001-0.150% (w/w);

farnesene isomer 1 0.001-0.150% (w/w);

caryophyllene oxide 0.001-0.150% (w/w);

alpha-bisabolol 0.001-0.150% (w/w);

beta-myrcene 0-0.005% (w/w);

gamma-terpineol 0-0.050% (w/w);

trans-nerolidol 0-0.050% (w/w);

In a preferred form, said composition comprises:

cannabidiol (CBD) 4,200-5,100% (w/w);

cannabigerol (CBG) 0.150-0.220% (w/w);

cannabichromene (CBC) 0.200-0.270% (w/w);

tetrahydrocannabinol (THC) 0.150-0.230% (w/w);

cannabidivarin (CBDV) 0.01-0.050% (w/w);

cannabidiolic acid (CBDA) 0.010-0.070% (w/w);

cannabinol (CBN) 0.010-0.050% (w/w);

cannabidiol-C4 (CBD-C4) 0.010-0.050% (w/w);

beta-caryophyllene 0.020-0.100% (w/w);

alpha-humulene 0.008-0.100% (w/w);

farnesene isomer 3 0.008-0.100% (w/w);

farnesene isomer 2 0.008-0.100% (w/w);

farnesene isomer 1 0.008-0.100% (w/w);

caryophyllene oxide 0.008-0.100% (w/w);

alpha-bisabolol 0.008-0.100% (w/w)

beta-myrcene 0-0.050% (w/w);

gamma-terpineol 0-0.050% (w/w);

trans-nerolidol 0-0.050% (w/w).

In an even more preferred form, said composition comprises:

cannabidiol (CBD) 4.600-5.050% (w/w);

cannabigerol (CBG) 0.170-0.210% (w/w);

cannabichromene (CBC) 0.205-0.250% (w/w);

tetrahydrocannabinol (THC) 0.170-0.240% (w/w);

cannabidivarin (CBDV) 0.020-0.040% (w/w);

cannabidiolic acid (CBDA) 0.020-0.060% (w/w);

cannabinol (CBN) 0.020-0.040% (w/w);

cannabidiol-C4 (CBD-C4) 0.015-0.040% (w/w);

beta-caryophyllene 0.040-0.090% (w/w);

alpha-umulene 0.010-0.080% (w/w);

farnesene isomer 3 0.010-0.080% (w/w);

farnesene isomer 2 0.010-0.080% (w/w);

farnesene isomer 1 0.010-0.080% (w/w);

caryophyllene oxide 0.010-0.080% (w/w);

alpha-bisabolol 0.010-0.080% (w/w) beta-myrcene 0.001-0.010% (w/w);

gamma-terpineol 0.001-0.010% (w/w);

trans-nerolidol 0.001-0.010% (w/w).

In a further aspect, a formulation for topical use is here claimed which comprises the composition according to the present invention and one or more excipients.

Preferably, said excipients are selected from the group which comprises: glycerin, glyceryl stearate, PEG-100 stearate, cetyl alcohol, allantoin, Butyrospermum parkii, tocopheryl acetate, Lavandula angustifolia oil, xanthan gum, juice of Aloe barbadensis leaves, triethanolamine, bisabolol, disodium EDTA, vitamin B3, binders, disaggregants, glidants, preservatives.

Said formulation optionally comprises further active ingredients selected in the group which comprises: keratolytic agents, anti-irritation agents, antioxidants, anti-redness agents of the skin, preservative, filler, emulsifier, humectant, thickener, nourishing agent for the skin, moisturizing agent for the skin, occlusives, emollient agents, calming agents, soothing agents.

In particular embodiments, said formulation for topical use comprises the composition according to the present invention and water, cetearyl alcohol, pentylene glycol, glycerin, caprylic/capric triglyceride, Prunus amygdalus dulcis oil, isoamyl laurate, Butyrospermum parkii butter, squalane, cetearyl glucoside, polyglyceryl-6 laurate, polyglyceryl-6 esters of olive oil, acryloyl dimethyl taurate/vp ammonium copolymer, hydroxyacetophenone, betaine, panthenol, tocopheryl acetate, tocopherol, allantoin, sodium lauroyl lactylate, Aloe barbadensis leaf juice, bisabolol, sodium hyaluronate, tetrasodium glutamate diacetate, ceramide np, ceramide ap, cholesterol, honokiol, magnolol, phytosphingosine, carbomer, xanthan gum, ceramide eop, and dimethicone.

In a further aspect, the composition according to the present invention is claimed for use in the prevention and/or dermatological and/or cosmetic treatment of damage to the mucous membranes, skin and skin adnexa.

In one embodiment, said composition is for use in the topical dermatological treatment of damage to mucous membranes, skin and skin adnexa such as, by way of example, acceleration of wound healing times, antibacterial action, treatment of herpes, psoriasis, vitiligo, atopic dermatitis, antioxidant and detoxifying action, barrier effect.

In a further aspect, cosmetic use is claimed for an anti-aging, moisturizing, lightening/brightening, anti-reddening effect.

In a further aspect, the food use of the composition according to the present invention is claimed. In particular, the use of the composition according to the present invention as a functional ingredient is claimed.

In one embodiment, said composition for food use is used to promote sleep, good mood and serenity; with calming effect; to promote digestion; to counteract nausea; to counteract localized and general tension; as an antioxidant; for skin protection; to promote intestinal homeostasis.

The following examples are intended to better describe the invention and are not intended to be in any way limitative of the same, the scope of which is defined by the following claims.

### EXAMPLES

### Example 1: Preparation of the Extracts of Cannabis sativa According to the Invention

Cannabis sativa and ethanol were loaded into a suitable container, and the heterogeneous solution was heated up to 78° C. under stirring.

At the end, the stirring was stopped and the ethanolic solution percolated into another container.

Ethanol was again added to the solid mass and the heterogeneous solution was again heated up to 78° C. under stirring.

At the end, the stirring was stopped and the ethanolic solution percolated into another container, combining it with the previous one.

The ethanolic extractions were repeated until exhaustion of the raw material.

The combined ethanolic extractions were concentrated up to a weight between 10% and 30% of their initial weight, the concentrated solution was brought to a temperature between 70° C. and 85° C. until the ratio of cannabidiol and the sum of cannabidiol and cannabidiol acid (CBDA) was not found to be 85%.

Finally, the ethanol was completely distilled and an amount of MCT oil was added such as to obtain the composition according to the present invention with the desired CBD title.

### Example 2: Qualitative and Quantitative Characterization of the Cannabis sativa Extract According to the Invention

In order to obtain a qualitative-quantitative characterization of the extract of the present invention, four batches of Cannabis sativa extract in medium chain triglycerides (MCT), obtained according to the procedure described in example 1 from plants coming from two different crops, in two different years, were analyzed by HPLC and GC analysis.

The cannabinoid analysis was carried out using an HPLC with UV/VIS detector and mass spectrometer as well as the corresponding standards for the cannabinoids to be researched, a validated analytical method has been used.

The terpenes were analyzed using a GC with flame ionization detector and a set of standards of terpenes, using a validated method that uses alpha-Pinene as a quantitative standard.

The cannabinoids sought are:

1. cannabidivarin (CBDV),

2. cannabidivarinic acid (CBDVA),

3. tetrahydrocannabivarin (THCV),

4. cannabidiol (CBD),

5. cannabigerol (CBG),

6. cannabidiolic acid (CBDA),

7. tetrahydrocannabinol (THC),

8. tetrahydrocannabinolic acid (THCA),

9. delta-8-tetrahydrocannabinol (TH8),

10. cannabigerolic acid (CBGA),

11. cannabinol (CBN),

12. cannabichromene (CBC) e

13. cannabidiol-C4 (CBD-C4)

The terpenes sought are:

1. alpha-Pinene,

2. Camphene,

3. Sabinene,

4. beta-Pinene,

5. beta-myrcene,

6. alpha-Fellandrene,

7. 3-Fairings,

8. alpha-Terpinene,

9. Limonene,

10. Eucalyptol,

11. cis-beta-Ocimene,

12. trans-beta-Ocimene,

13. gamma-Terpinene,

14. Sabinene hydrate,

15. Fencione,

16. Terpinolene,

17. Linalool,

18. Fenciolo

19. Camphor,

20. Isopulegolo,

21. Isoborneol,

22. Borneolo,

23. Menthol,

24. alpha-Terpineol,

25. gamma-Terpineol,

26. Nerolo,

27. Pulegone,

28. Geraniol,

29. Geranyl Acetate,

30. alpha-Cedrene,

31. beta-Caryophyllene,

32. Farnesene isomer 4,

33. alpha-Umulene,

34. Farnesene isomer 3,

35. Valencene,

36. Farnesene isomer 2,

37. cis-Nerolidol,

38. Farnesene isomer 1,

39. trans-Nerolidol,

40. Karyophyllene oxide,

41. Trouble,

42. Cedrolo,

43. alpha-bisabolol.

The qualitative and quantitative results obtained are summarized in the following tables:

TABLE 1
cannabinoids, (% w/w)

| | Quantità % | | | | | | | |
|---|---|---|---|---|---|---|---|---|
| Lotto | CBDV | CBD-C4 | CBD | CBG | CBDA | CBN | THC | CBC |
| CM5 74719004 | 0.019 | 0.020 | 4.836 | 0.138 | 0.020 | 0.001 | 0.222 | 0.235 |
| CM5 74719005 | 0.026 | 0.032 | 5.017 | 0.213 | 0.058 | 0.026 | 0.226 | 0.232 |
| CM5 74720001 | 0.026 | 0.024 | 4.856 | 0.225 | 0.016 | 0.009 | 0.212 | 0.216 |
| CM5 74720004 | 0.030 | 0.020 | 4.535 | 0.195 | 0.098 | 0.007 | 0.212 | 0.196 |
| Media | 0.025 | 0.024 | 4.811 | 0.193 | 0.048 | 0.011 | 0.216 | 0.220 |
| dev. Standard | 0.005 | 0.006 | 0.201 | 0.039 | 0.038 | 0.011 | 0.007 | 0.017 |
| RSD | 18.11 | 23.57 | 4.18 | 19.99 | 79.86 | 99.72 | 3.27 | 7.73 |

TABLE 2
terpenes, (% w/w)

| | CM5 74719004 | CM5 74719005 | CM5 74720001 | CM5 74720004 | media | Std. Dev | RSD |
|---|---|---|---|---|---|---|---|
| beta-myrcene | — | — | — | 0.005 | 0.005 | n.a. | n.a. |
| gamma-terpineol | 0.031 | — | — | — | 0.031 | n.a. | n.a. |
| beta-caryophyllene | 0.050 | 0.063 | 0.069 | 0.068 | 0.082 | 0.008 | 13.936 |
| alpha-humulene | 0.012 | 0.018 | 0.018 | 0.017 | 0.016 | 0.003 | 16.054 |
| farnesene isomer 3 | 0.015 | 0.020 | 0.021 | 0.020 | 0.019 | 0.003 | 13.443 |
| farnesene isomer 2 | 0.007 | 0.024 | 0.021 | 0.023 | 0.019 | 0.008 | 41.024 |
| farnesene isomer 1 | 0.010 | 0.031 | 0.028 | 0.030 | 0.025 | 0.010 | 39.348 |
| trans-nerodiol | — | 0.005 | 0.005 | 0.005 | 0.005 | 0.000 | 2.192 |
| caryophyllene oxide | 0.015 | 0.017 | 0.015 | 0.014 | 0.015 | 0.001 | 9.616 |
| alpha-bisabolol | 0.011 | 0.030 | 0.024 | 0.024 | 0.022 | 0.008 | 36.359 |
| Terpeni totali | 0.153 | 0.205 | 0.202 | 0.204 | 0.191 | 0.026 | 13.413 |

As can be clearly seen from the above data, the values of the prevalent chemical compounds are constant between the different batches analyzed, even when they come from plants harvested in different years.

In particular, it is noted that the cannabinoids most present in addition to CBD (about 5%) are: CBG, CBC and THC, each having a concentration of about 0.2%.

The overall content of terpenes is between 0.15 and 0.20%.

In particular, it is possible to identify 7 terpenes particularly characteristic of the extracts according to the present invention, namely: beta-caryophyllene, alpha-humulene, farnesene isomer 3, farnesene isomer 2, farnesene isomer 1, caryophyllene oxide, and alpha-bisabolol.

### Example 3: Cytotoxicity of the Cannabis sativa Extracts According to the Present Invention

The cytotoxicity tests were performed using the MTT assay, capable of measuring the mitochondrial succinic dehydrogenase enzyme, whose activity indicates cell viability, on human keratinocytes or fibroblasts incubated with the Cannabis sativa extract according to example 2 and with pure CBD. The integrity of the morphology before and after 6 and 24 hour incubation was assessed by light microscopy.

HaCaT cells, spontaneously immortalized human keratinocyte line (Boukamp et al., 1988) and normal human dermal fibroblasts (HDF) were cultured in DMEM (Gibco, Life Technologies, Monza, Italy) supplemented with 10% heat inactivated fetal bovine serum (Euroclone SpA, Milan, Italy), L-glutamine (2 mM; Gibco, Life Technologies, Monza, Italy), penicillin (100 U/ml), and streptomycin (100 mg/ml; Gibco, Life Technologies, Monza, Italy), at 37° C. in a humidified atmosphere containing 5% CO₂. Every 4 days, at 80-90% confluence, the cells were detached using 0.25% trypsin-EDTA (Gibco, Life Technologies, Monza, Italy), counted and placed in a new flask at a density of 1.5×10⁶ cells per flask, to allow for growth. For the purpose of the present analyses, the cells were seeded in 24-well plates and incubated with the extract according to the present invention or pure CBD at increasing concentrations.

After incubation, the culture medium was removed from each well, and 200 μl of a solution of 3,4,5-dimethylthiazol-2-yl-2,5-diphenyltetrazolium bromide was added for 30-40 minutes until the development of a purple color (formazan). Then 200 μl of an isopropanol:DMSO 90:10 solution was added to each well to extract the formazan from the cells. The absorbance was read by spectrophotometry at 570 nm (Envision, PerkinElmer, USA).

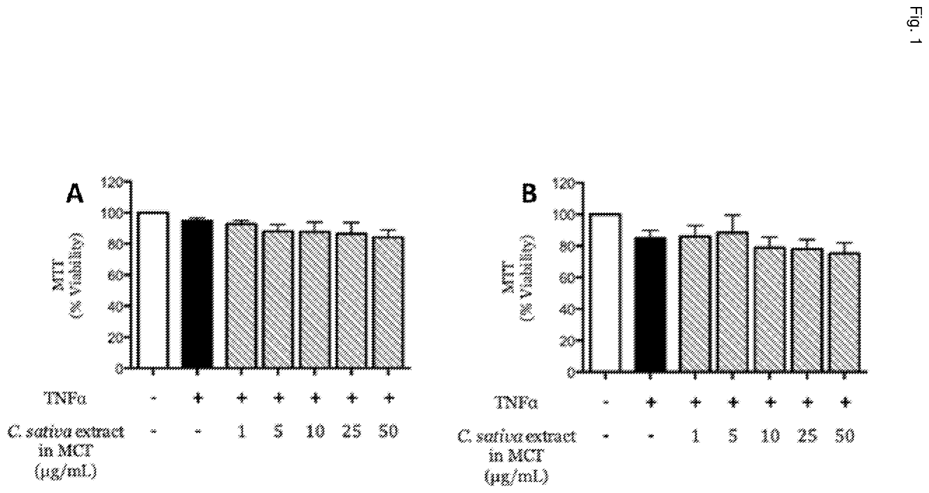

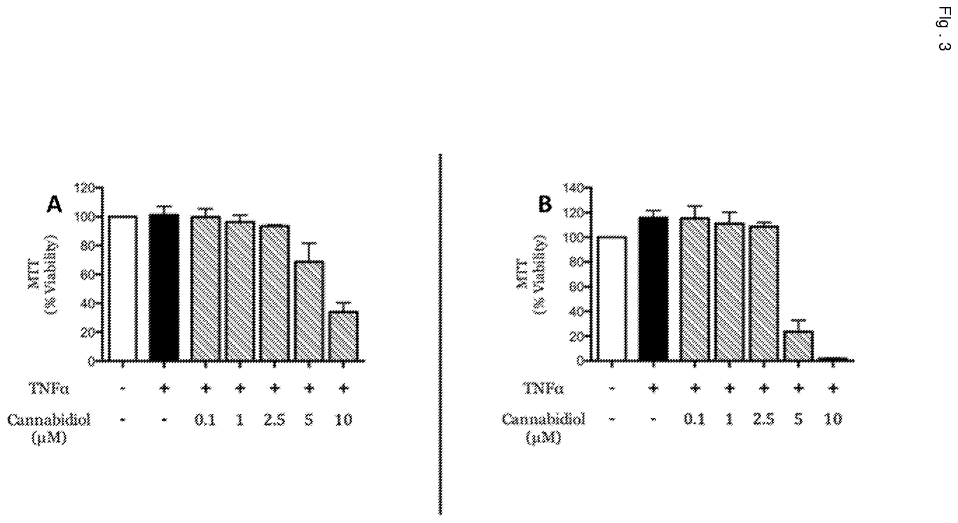

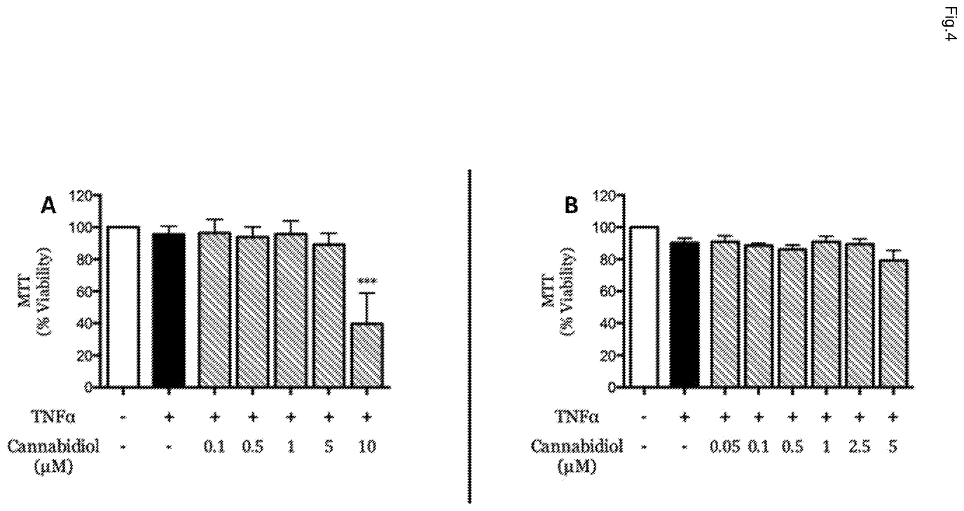

The extract according to the present invention showed no cytotoxic effects at any of the tested concentrations (1-50 μg/mL) after 6 hours (FIG. 1A, 2A) or after 24 hours (FIG. 1B, 2B) on both tested cell lines. In contrast, CBD showed cytotoxic effects in HDF cells already at concentrations above 2.5 μM (FIG. 3) and in HaCaT cells at concentrations above 5 μM (FIG. 4).

### Example 4: Anti-Inflammatory Activity of the Extracts of Cannabis sativa According to the Present Invention

To evaluate the anti-inflammatory activity of the extract according to the present invention, the in vitro effect after exposure to pro-inflammatory stimuli on the expression levels of factors known to be involved in inflammatory processes was evaluated.

The nuclear transcription factor NF-kB is activated by pro-inflammatory stimuli and activates the transcription of genes that amplify the inflammatory process, such as MMP-9, cytokines and growth factors such as VEGF.

In particular, the following were chosen as inflammation markers:
IL-8, a cytokine secreted by keratinocytes and fibroblasts following inflammation and particularly involved in skin diseases such as psoriasis;
MMP-9, a metalloprotease which degrades the extracellular matrix and which is activated following chronic skin inflammations such as dermatitis and psoriasis;
VEGF, the release of which by keratinocytes leads to the formation of the classic psoriatic plaques in patients suffering from this pathology.

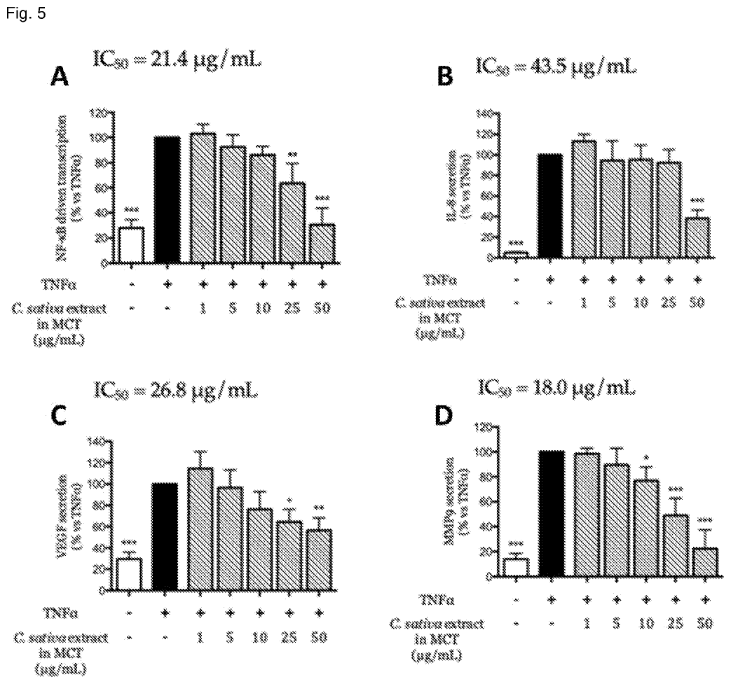

FIG. 5 shows the effect of the extract according to example 2 in human keratinocytes following treatment with the pro-inflammatory cytokine TNF-α (10 ng/ml) on NF-kB induced transcription (panel a), and on the expression levels of markers IL-8 (panel b), VEGF (panel c) and MMP-9 (panel d).

The data show a clear dose-dependent decrease in transcription induced by NF-kB (IC50=21.4 μg/mL), as well as in the expression levels of the selected markers following exposure to the extract according to the present invention. It is important to observe how the effect is present at doses of the extract that do not show any toxicity on the cells.

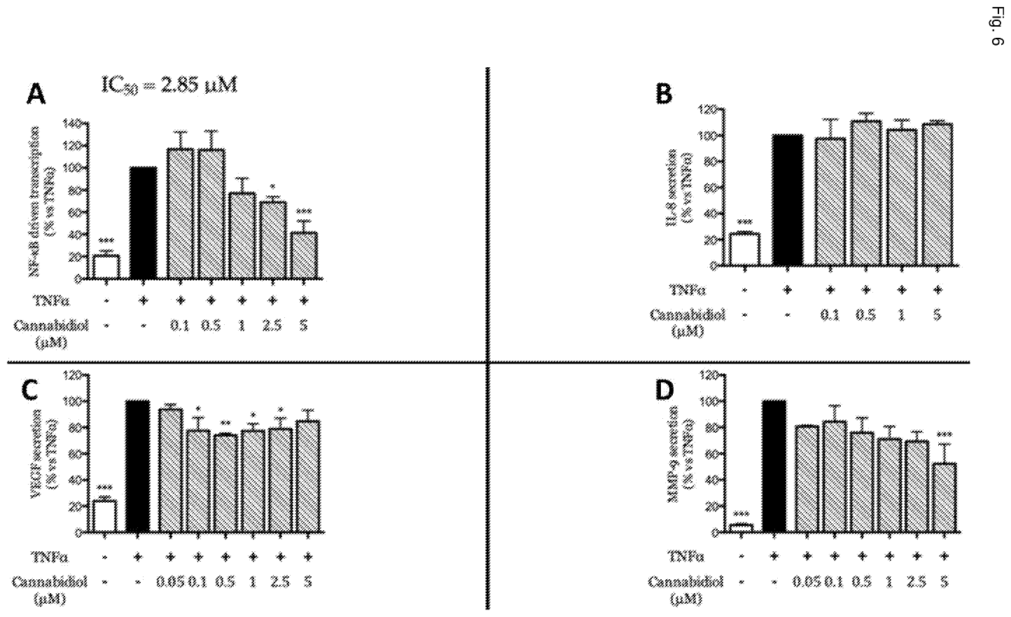

FIG. 6, by way of comparison, shows the effects of pure CBD on the same inflammatory markers previously considered for the extract. In human keratinocytes, CBD inhibits NF-kB-driven transcription in a concentration-dependent manner, with an IC50 of 2.85 μM. However, contrary to what is shown for the extract according to the present invention, CBD has no effect on the expression levels of IL-8, while the effect on MMP-9 and VEGF is modest.

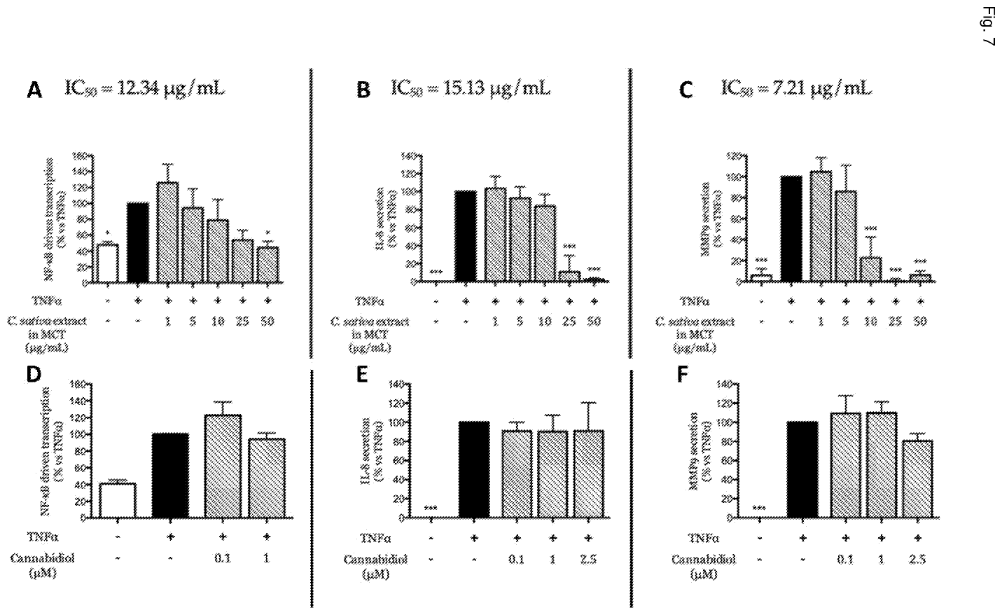

FIG. 7 reports the results obtained in human fibroblasts.

Similarly to what has been observed in keratinocytes, the extract inhibits the release of IL-8, MMP-9 and NF-kB-guided transcription in a concentration-dependent manner, with a lower IC50, therefore with a greater inhibitory power on the release of IL-8 and MMP-9 with respect to the effect on human keratinocytes.

In contrast, CBD showed no effect on the release of IL-8 and MMP-9, nor is any effect on NF-kB noted.

As a negative control, the cells were incubated with a solution of MCT, inert vehicle of the composition according to the present invention, without observing any effect.

1. A composition, comprising:
cannabinoids in a concentration of between 0.500 and 10.000% (w/w), and
terpenes in a concentration of between 0.005 and 1.000% (w/w).

2. The composition according to claim 1, comprising:
the cannabinoids in a concentration of between 4.000 and 7.000% (w/w), and
the terpenes in a concentration of between 0.050 and 0.300% (w/w).

3. The composition according to claim 1, comprising:
from 0.5 to 6.9% (w/w) of cannabidiol (CBD) as a cannabinoid,
from 0.1 to 3% (w/w) of other cannabinoids different from CBD, and
from 0.005 to 0.5% (w/w) of the terpenes.

4. The composition according to claim 3, comprising:
from 4 to 6% (w/w) of the cannabidiol (CBD),
from 0.2 to 2% (w/w) of the other cannabinoids different from CBD, and from 0.05 to 0.3% (w/w) of the terpenes.

5. The composition according to claim 1 obtained by extraction of Cannabis sativa L.

6. The composition according to claim 5, wherein the extraction is carried out on Cannabis sativa L. varieties having an average content of tetrahydrocannabinol (THC) below 0.2%.

7. The composition according to claim 1, wherein the cannabinoids are selected from the group consisting of cannabidiol (CBD), cannabigerol (CBG), cannabichromene (CBC), tetrahydrocannabinol (THC), cannabidivarin (CBDV), cannabidiolic acid (CBDA), cannabinol (CBN), and cannabidiol-C4 (CBD-C4).

8. The composition according to claim 1, wherein the terpenes are selected from the group consisting of beta-caryophyllene, alpha-humulene, farnesene isomer 3, farnesene isomer 2, farnesene isomer 1, caryophyllene oxide, and alpha-bisabolol.

9. The composition according to claim 1, in the form of ethanolic extract.

10. The composition according to claim 1, further comprising an inert oily carrier.

11. A formulation comprising the composition of claim 1 and one or more excipients.

12. The formulation according to claim 11, wherein the one or more excipients are selected from the group consisting of glycerine, glyceryl stearate, PEG-100 stearate, cetyl alcohol, allantoin, Butyrospermum parkii, tocopheryl acetate, Lavandula angustifolia oil, xanthan gum, Aloe barbadensis leaf juice, triethanolamine, bisabolol, disodium EDTA, vitamin B3, a binder, a disaggregant, a glidant, and a preservative.

13. A method of treating a mucous membrane, skin, or skin adenexa, the method comprising:
contacting the mucous membrane, skin, or skin adenexa by topical application with the formulation of claim 11.

14. The method of claim 13, wherein the topical application prevents mucosal, skin, and/or skin adnexa damage, and/or the topical application is a dermatologic and/or cosmetic treatment of mucosal, skin, and/or skin adnexa damage.

15. A foodstuff, comprising the formulation of claim 11.
